(19) United States
(12) Patent Application Publication
(10) Pub. No.: US 2018/0189688 A1
(43) Pub. Date: Jul. 5, 2018

(54) METHOD FOR USING THE CAPACITY OF FACILITES IN A SKI AREA, A TRADE FAIR, AN AMUSEMENT PARK, OR A STADIUM

(71) Applicant: SKIDATA AG, Grödig/Salzburg (AT)

(72) Inventors: Martin HAIDACHER, Salzburg (AT); Thomas SCHLECHTER, Seekirchen am Wallersee (AT)

(21) Appl. No.: 15/825,685

(22) Filed: Nov. 29, 2017

(30) Foreign Application Priority Data
Dec. 29, 2016 (EP) 16207352.2

(51) Int. Cl. G06Q 10/04; H04W 4/02; H04L 29/08; A61L 9/14; G06Q 30/02

(52) U.S. Cl. CPC G06Q 10/04; H04W 4/027; G06Q 30/0267; H04L 67/306; A61L 9/14; H04W 4/023

(57) ABSTRACT

A method is proposed for using the capacity of a facility at a ski area, a trade fair, an amusement park, or a stadium, within the scope of which the current position and the motion profile of all persons present in a region of the facility of the ski area, the trade fair, the amusement park, or the stadium is detected. A people map is provided on a server which corresponds to the spatial distribution of the persons, and by the spatial distribution of persons as a function of time, a motion profile of the persons is provided. Depending on the current position of the present persons, the motion profile of the present persons, and the current use of the facilities, all or individual persons obtain information generated by the server that can effect a redirection of the persons, so that use of a capacity of the facility can be optimized.

This application claims priority form European patent application serial no. 16207352.2 filed Dec. 29, 2016.

### FIELD OF THE INVENTION

The present invention relates to a method for using the capacity of facilities in a ski area, trade fair, amusement park, or a stadium.

### BACKGROUND OF THE INVENTION

In ski areas, trade fairs, amusement parks, or stadiums, as a rule a plurality of facilities are provided; for example, a ski area comprises several trails with the corresponding conveying facilities in the form of lifts, restaurants, huts, etc.

Here it is often the case that with some facilities there are long lines or large accumulations of persons, so that on the one hand the comfort of persons is impaired, and on the other, the capacities of the other facilities are not fully utilized; for example, in a ski area there can be lines formed in front of restaurants, while the capacity of the ski lifts is not fully utilized.

### SUMMARY OF THE INVENTION

The problem to be the solved is to provide a method for using the capacity of facilities in a ski area, a trade fair, or an amusement park.

This problem is solved by the features of the independent claim(s).

Therefore a method for using the capacity of facilities in a ski area, a trade fair, an amusement park, or a stadium is proposed, in the scope of which the current position and motion profile of all of the persons present in the region of a ski area, a trade fair, an amusement park, or a stadium are detected, wherein in a server a people map is provided that corresponds to the spatial distribution of the persons, wherein through the spatial distribution of persons as a function of time, a motion profile of the persons is provided, and wherein depending on the current position of the present persons, the motion profile, and the current use of the facilities, all or individual persons receive information generated by a server, which can effect a redirection of said persons so that use of capacity of the facilities can be optimized.

The detection of persons can be effected according to a first embodiment of the invention by means of mobile electronic devices, e.g. mobile telephones, which the persons carry with them, via a standard for communicating in radio networks. For example, the mobile telephones of the persons can transmit via a radio standard, e.g. Bluetooth Low Energy-Standard (BLE) or via a WLAN standard within the scope of “broadcasting,” transmitting an unambiguous ID at regular intervals, which can correspond to the IMEI number of the device, which is received by transceiver units connected to a server, e.g. BLE transceiver units or WLAN transceiver units in the range of the mobile electronic device. The radio standard can for example also be the UWB standard (ultra wide band) for close-range communication, which allows localization of the mobile electronic devices with greater accuracy.

Hereby the localization of the mobile electronic devices is implemented in that with reference to a reference signal intensity for the radio standard used, which as a rule corresponds to the signal intensity 1 m distant and which is contained as information in the signal of the mobile electronic device or, depending on the mobile electronic device, for example on the type of mobile telephone, is contained in a table stored in the computer, the distance between the mobile electronic device and the transceiver located within range is obtained for the respective radio standard, wherein subsequently a trilateration or for more than three transceivers, a multilateration can be carried out.

Alternatively, the localization of mobile electronic devices can be effected in that they transmit the current GPS data obtained by means of a GPS module integrated in the mobile electronic devices along with the unambiguous ID via the utilized radio standard to the transceivers located within range.

In the scope of a further development, the mobile electronic device, by means of which the current position of all persons present in the region of the ski area, trade fair, or the amusement park is detected, a portable apparatus is to be used that is provided to every person on entry if they do not have such an apparatus. This apparatus comprises a component that permits communication with the transceivers of the ski area, trade fair, or amusement park by means of a radio standard, and optionally a display apparatus and a component for issuing acoustic signals. The portable apparatus is issued to each person.

Further, the apparatus can have a GPS module, by means of which, on the one hand, the precise position of the apparatus, and on the other the direction of motion and speed at which the apparatus is moving, are detected. For the case of a ski area, the apparatus is designed such that it may be attached to one of the skis or a snowboard such that the display device is visible to the user when skiing; hereby, if no information generated by the server is displayed, the display device can show the speed, the height, the traveled meters in height, etc. Further, the device can be coupled to a mobile electronic device of the user via the radio standard, wherein the user can be informed when for example skis left in front of a restaurant are moved, implementing a simple theft protection.

Within the scope of a further embodiment, which is suited in particular for amusement parks, the portable apparatus can be configured as AR/VR glasses (augmented reality/virtual reality glasses).

In accordance with a further embodiment of the invention, persons can be detected and localized by means of a 3D camera, for example TOF camera, meaning cameras that measure distances by means of a time of flight (TOF) procedure, stereo cameras or PMD cameras, namely TOF cameras comprising a photo mix detector, which by means of image analysis permit precise position detection of persons. The cameras are connected to the server. Detection by means of cameras can also be carried out by means of mobile electronic devices to increase the localization precision in this manner. A person detected and localized by means of a mobile electronic device at the entrance to a ski area, a trade fair, or an amusement park can accordingly also be detected by means of the camera, by which the person can be assigned person-specific information, for example age and sex.

Furthermore, trinocular cameras can be used, each of which comprises three individual cameras of different distances from one another, which are arranged in a series, wherein in each case two of these individual cameras act as a stereo camera. Advantageously, the different combination possibilities of the individual cameras to form a stereo camera permit a broad variation of the possible focal distances of the temporary general system and thus an optimal analysis of the areas of interest (AoI).

Here a stereo image, which also contains depth information regarding the persons and objects located in the scope of consideration, supplies a suitable assessment, for example using the well-known census transform and combination of images of each individual camera. The assessment can hereby be made in the camera itself or else in a computer assigned to the camera. Depending on the configuration of the computer components, by optimization of the parameters resolution/mono-image rate/stereo-image rate/selected algorithm/permitted delay (real-time capacity) different performance demands on the system are possible, which permits an optimal and efficient adjustment of the system in the field to the respective circumstances.

The obtained depth information can be further used in a next step to isolate persons in the image, thus to detect their presence and ultimately to be able to implement a position determination. Here numerous, diverse methods are known to a person skilled in the art from the literature, for example methods that are based on the analysis and extraction of textures in an image; methods that are based on analysis and interpolation of motion via several sequential images; as well as model-based approaches, which can be implemented in the context of person identification either monolithically, thus by analyzing the human body as a whole, or parts-based, thus recognizing individual parts of the human body and assembling these into a whole human body according to suitable hypotheses. The latter approach is suited in particular for the case when a person in the image is partially obscured.

According to one embodiment of the invention, in a next step within the scope of a training phase, typical partial contours of the objects to be examined are derived from image sequences and stored in a data bank. For this purpose, the textures in the image to be examined can first be highlighted for example with anisotropic filters, which typically are used in 3D analysis methods and are well known in the literature. The advantage of the method of texture recognition is that depending on the field of application, different templates can be provided that optimize the results; for example, for an access control system of a ski area templates containing a helmet are provided. Thus, there exists an optimal set of detection parameters for every application case. Since the produced textures are simple, they can also be easily subjected to transformations in real time, such as for example compressions, rotations, or elongations. Thus, these textures in the image can also be recovered if the person turns or the distance to persons changes.

In a next step, a further set of information can be produced from the image, which can be obtained by the so-called “approximate texture context” analysis. Here small, low-complexity 2D structures can be extracted from the images. Structures of this type typically occurring in human bodies are accordingly filed in a code book. Hereby information regarding mutual local correlation in the image between the individual structures can also be stored. Using suitable statistical methods, subsequently these individual structures can be tested with respect to their mutual positions. If this test shows that based on the position of the structures with respect to one another and the individual properties of the structures it is plausible that a set of these structures describes a human person, this subset is characterized as such and the corresponding image area receives a (virtual) bounding box to indicate the presence of a human person.

In a further step, the above approaches can be combined in that for example a maximum a posteriori assessment method is applied that is well known to the skilled person. The result provides a robust valuator for the individual position as well as positioning (e.g. posture, rotation, head turning, arm direction) of the persons in the image.

Given knowledge of the surroundings and the camera location, this information can be subsequently converted to absolute x-y-(z) coordinates in the real surroundings, which can subsequently be used as the basis for localization, counting, and tracking of persons.

By means of 3D or trinocular cameras a person detection and recognition can accordingly be carried out.

Accordingly, persons in a ski area, a trade fair, or an amusement park can be detected in real time or at—for example predetermined—time intervals so that a people map is provided in a server that corresponds to the spatial distribution of persons. By means of the spatial detection of persons as a function of time, a motion profile of the persons is provided. With reference to the unambiguous ID transmitted by the mobile electronic devices and/or with reference to the data of 3D or trinocular cameras, the detected persons can be tracked.

By the assessment of the people map it is possible in the ski area, in the trade fair grounds, or in the amusement park to predict and prevent the formation of queues or large crowds. This comes from an analysis of current and if available known older motion profiles of individual persons as well as the totality of persons with reference to the people map. Depending on the current position of the present persons and the current use of the devices, all or individual persons can receive information generated by a server that can effect a redirection of these persons. The information is preferably person-related.

The motion profile can contain the average speed and direction of motion at places that require a decision regarding the direction of motion with respect both to individual persons and to person groups. Furthermore, the motion profiles can contain the durations of stay of individual persons or groups at specific places, in particular in front of advertising signs, information signs and the like. Furthermore, person-specific information can be assigned to a person, for example information about age group or sex that can be obtained by assessment of the images from 3D or trinocular cameras. The motion profiles and if available the person-specific information about a person are assigned in the server to a detected person and thus to an unambiguous visitor profile. An unambiguous visitor ID is assigned to each visitor profile.

For the case when the detection of persons by means of mobile electronic devices, for example mobile telephones that the persons carry occurs via a standard for communication in radio networks, wherein the devices transmit an unambiguous ID at regular intervals, 3D or trinocular cameras can be provided at the entrances, the images of which are assessed with regard to person-specific information, linked to the ID of the respective mobile electronic device and assigned to a user profile.

The user profiles and if available person-specific information are assessed to predict the future motion profile of persons and groups for a specific predetermined time interval. For example, if a person remains for a long time in front of an advertising poster, there is a high likelihood this person will visit the advertised facility within the next 30 minutes. However, if this person has visited a food stand prior to this, there is a high probability he/she will visit the facility within 15 minutes since other food stands on the way there will be ignored.

The motion profiles for persons and/or person groups can be obtained during the current visit of the persons group, wherein the quality of the data is improved over time during the visit day. Furthermore, typical modes of behavior of all visitors for one day can be evaluated to detect the motion profiles depending on external factors such as the weather for example.

In accordance with the invention, depending on the results of the assessment of the motion profile, measures can be taken to avoid the formation of queues. For example, another checkout can be opened or persons can be redirected by messages containing suitable advertisements or information to avoid the formation of queues at specific sites and to utilize the capacity of additional facilities.

In the scope of a further embodiment of the invention, visitors can be proactively informed of offerings, wherein this preferably occurs by a transmission of push messages to the mobile electronic devices by means of the utilized radio standard.

If an already available visitor profile can be assigned to a detected person with reference to the ID transmitted by the mobile electronic device or with reference to the data about 3D or trinocular cameras, that is if it is a regular visitor, it is tested in the server whether motion profiles for this visitor profile exist from the long-term past; if this is the case, it is tested whether a motion profile from the short-term past exists, i.e. from the current day. If a motion profile of from the short-term past exists, it is tested whether an assessable context from the current visit is available, which is the case for example if the visitor is there with children. If this is not the case, the motion profiles from the long-term and the short-term past are combined to make a prediction for the short-term future, that is for a specific predetermined time interval in the future. For the case that an assessable context exists, this is combined with the motion profiles from the short-term and the long-term past to improve the prediction.

If no motion profile from the short-term past exists, and assessable context from the from the current visit exists, the motion profiles from the long-term past are combined with the assessable context to make the prediction. If no assessable context exists, the motion profiles from the long-term past are used to make the prediction.

For the case when no motion profile from the long-term past exists and a motion profile from the short-term past and/or and assessable context exist, the available information is used for prediction of the short-term future. If no motion profile exists from the long-term and from the short-term past, the current position information is stored to update the motion profile information.

The stored position information is combined into a motion profile, wherein actions are derived from the prediction results in order to guide the visitor in such a way that no congestion or queues occur, the capacity of the facilities is used, and the needs of the visitor are optimally met.

If no visitor profile can be assigned to the detected person if the people map, a new visitor profile is filed and assigned to this person. This visitor profile is linked to the ID of the mobile electronic device which the person carries and/or to the images of 3D or trinocular cameras.

The approach corresponds to the described approach for the case when a visitor profile already exists in the databank except that there are no motion profiles the past.

### BRIEF DESCRIPTION OF THE DRAWINGS

The features of the invention are discussed with reference to the accompanying figures in which:

FIG. 1 shows a schematic of a server; and

FIG. 2 shows a chair of a chairlift offering places for six people in which a display device is provided for each seat.

### DETAILED DESCRIPTION OF THE PREFERRED EMBODIMENTS

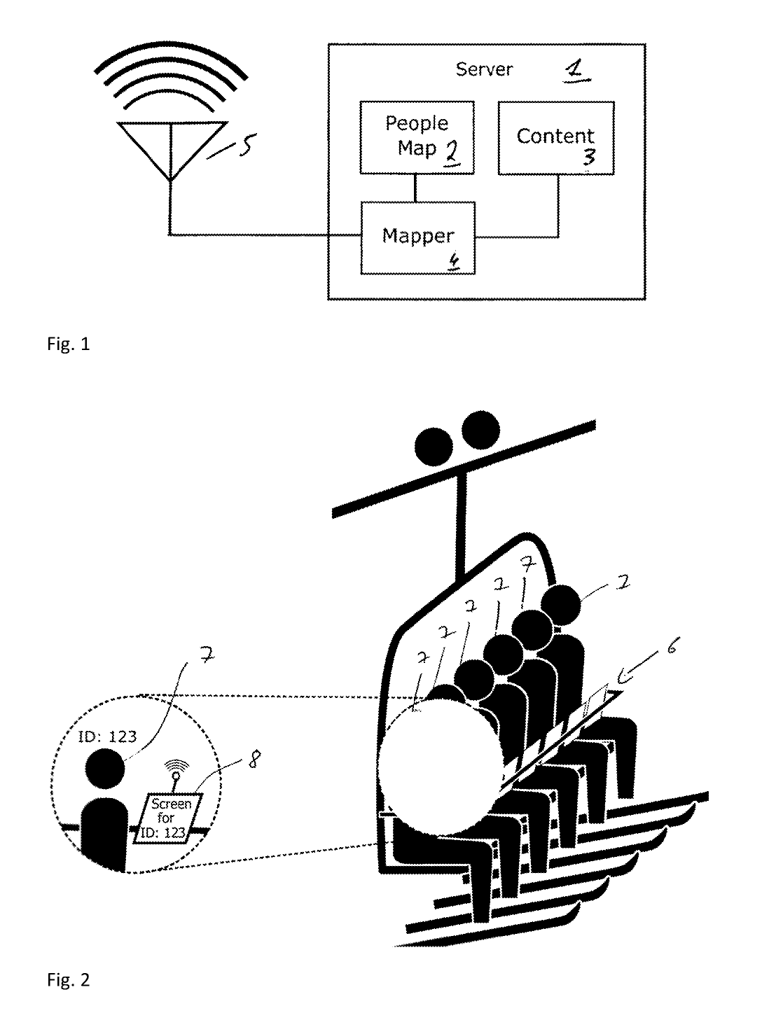

With reference to FIG. 1, an embodiment of the method explained by way of examples. In FIG. 1 a schematic of a server 1 is shown. According to the invention a people map 2 is provided in the server 1, which people map corresponds to the spatial distribution of persons, wherein by means of the spatial detection of persons as a function of time, in server 1 a motion profile of the persons is provided. Furthermore, in the server and information (content) databank 3 containing the visitor profiles, wherein by means of a mapping module (mapper 4), depending on the current position of the persons present, the visitor profiles, and the current use of the facilities, all or individual persons receive generated information that is transmitted via a suitable communication interface 5.

The information or the measures for guiding the persons can be push messages or text messages to a mobile electronic device of the respective person and/or the control of dynamic context-sensitive billboards depending on the current position of specific persons or groups. In the scope of a further development, the context-sensitive billboards can have a touch screen, wherein the person or group of persons standing in front of the Billboard can be encouraged by means of acoustic and/or optical signals, for example by means of blinking points to click on the touchscreen on specific areas of the touchscreen in order to then receive messages there, the content of which can influence the behavior of the person or the group in a specific manner.

For the case when the persons in accordance with the invention receive push messages, the latter can be transmitted by means of the radio standard used to detect the persons or via WLAN, preferably in combination with a specific app. The specific contents display of the messages can be in text form, on the basis of context-based geographic maps, or in the form of a specific navigation implementation by means of the specific app.

In addition, the push messages comprising information, advertising overlays, and never occasional instructions can be displayed on VR/AR glasses.

In the scope of a further development of the invention, the information that can effect a redirection of these persons can be combined with a geocaching application implemented in the portable electronic device, wherein the persons receive bonus points if they approach attractions in a dynamically definable sequence.

The redirection of the users can alternatively or additionally also the optical by means of a multicolor LED arrangement embedded in the ground within a limited area or by means of light projections on the ground, preferably by means of laser projections, wherein depending on the position of individual persons or groups of persons, individual persons or groups can be guided from the directions of the LED arrangement or the laser projections to the facilities. For example, by means of the active LEDs or the projections, arrows are formed, by means of which a specific direction is displayed.

Furthermore, a special color can be assigned to each detected person, wherein this can be done for example by means of a colored armband that persons receive at the entrance, by blinking of status LEDs in a specific color on the mobile electronic device. Here a person or a group can be guided by colored signals in their surroundings in the desired manner.

Alternatively, or additionally, microdrones can also be used that are assigned to selected persons or groups of persons permanently or dynamic. The microdrones accompany the persons or groups either for the entire day or else the persons or groups of persons as needed can be dynamically assigned to a microdrone by blinking of the OD and position of the persons or group of persons to the position of a microdrone. The microdrones can hereby be equipped with acoustic and/or visual signaling elements; furthermore, in combination with VR/AR glasses, the assignment to a person or group of persons can also be shown in the display of the VR/AR glasses, wherein with the existing assignment an audio playback can be carried out by means of the VR/AR glasses during viewing of the microdrone.

In the scope of a further embodiment, which is especially suited for amusement parks, by means of portable apparatus in the form of AR/VR glasses (augmented reality/virtual reality glasses), assigned to each person, the respective person is shown virtual signs, instructions, and or virtual assistants. Furthermore, a geocaching app can be provided in the AR/VR glasses, by means of which the respective person can obtain bonus points in a customer loyalty program if they approach facilities in a dynamically definable sequence.

In the case of a ski area comprising chairlifts, according to one embodiment of the invention it is proposed that a display apparatus be provided for each seat in a chairlift, over which messages are displayed to the respective person during the ride which can serve to redirect this person. If for example at the current time there are still places open in a restaurant of the ski area and the person has not yet visited a restaurant, information can be displayed to this person about this restaurant and optionally special offerings, which also can be about receiving bonus points when visiting this restaurant if the person visits the restaurant within a predetermined time. Furthermore, the person can be shown alternatives with regard to ski slopes which at the current time can be visited with no queues at the lifts. The content of the messages preferably is determined depending on the motion profile of the respective person. If the person based on the motion profile largely skis the red qualified slopes, the person is shown the corresponding alternatives.

This embodiment is illustrated with reference to the enclosed FIG. 2. FIG. 2 shows a chair 6 of a chairlift offering places for six persons 7, wherein for each seat a display device 8 provided. With reference to the people map, the sitting position of the persons 7 is known so that all or individual persons 7 can be shown a message over the respective display apparatus 8, which message depends on the prospective visitor profile. In the example shown, the person with the visitor ID 123 receives a message specific to him/her.

In the scope of a further development of the invention, a fragrancing device can be provided for each seat in a chairlift which, if a display apparatus is provided, can also be integrated into the display apparatus. By means of the fragrancing device, fragrances can be sprayed that can serve to redirect this person, and for the case when a display apparatus is provided, can correspond to the messages displayed over the display apparatus; for example, during the display of free places in a restaurant, a corresponding fragrance can be sprayed; if they wellness facility is displayed with the purpose of redirecting the person to this facility, a fragrance of ethereal oils can be sprayed.

For the case of a cable car, a display apparatus and/or a fragrancing device can be provided for each gondola, wherein in this case the messages that can serve to redirect this person are aimed at all passengers.

In accordance with the invention, for the case of a ski area comprising ski lifts and access control facilities to the lifts, at each access control facility to the lifts of the ski area a display apparatus can be provided, wherein by means of the display apparatus messages can be displayed to the person standing in front of the display apparatus during the ride which can serve to redirect this person; the content of the messages is determined preferably depending on the motion profile of the respective person.

1-15. (canceled)

16. A method for using a capacity of a facility at a ski area, a trade fair, an amusement park, or a stadium, wherein a current position and motion profile of all persons present in a region of the facility of the ski area, trade fair, amusement park, or stadium are detected,
generating a people map on a server which corresponds to a spatial distribution of the persons present in the region,
providing, via the spatial distribution of the persons as a function of time, a motion profile of the persons, and
depending on the current position of the persons present, the motion profile of the persons present, and a current usage of the facility, one or more individual persons receiving information, generated by the server, which can effect redirection of these persons so that the capacity of the facility can be optimized.

17. The method according to claim 16, further comprising transmitting an unambiguous ID, via a mobile electronic device that each of persons carries, via a standard for communicating in the radio network in a scope of “broadcasting,” which is received by transceivers connected to a server within a range of the mobile electronic device, for detecting the persons,
effecting localization of the persons by the mobile electronic devices transmitting current GPS data, obtained by a GPS module integrated into the mobile electronic device, along with the unambiguous ID via the used radio standard to the transceivers located within range, localizing the persons by person identification using 3D or trinocular cameras, or with reference to a reference signal intensity for the used radio standard, which is contained as information in the signal of the mobile electronic device, or depending on the mobile electronic device, is filed in a table stored in the computer, a distance between the mobile electronic device and the transceivers located within range is obtained for the respective radio standard, and
subsequently carrying out a trilateration or, for more than three transceivers, a multilateration.

18. The method according to claim 17, further comprising using a Bluetooth Low Energy Standard (BLE), a WLAN standard, or a UWB standard as the standard for communication in radio networks, and the unambiguous ID corresponds to an IMEI number of the mobile electronic device.

19. The method according to claim 17, further comprising detecting, via the mobile electronic device, a current position of all persons present in the region of the facility of the ski area, the trade fair, or the amusement part, and
giving a portable apparatus to each person, if such person does not have a portable apparatus, which comprises a component that permits communication with transceivers of the facility of the ski area, the trade fair, or the stadium by a radio standard, and optionally a display device, a component for output of acoustic signals, and/or a GPS module, such that a portable apparatus is assigned to each person.

20. The method according to claim 19, further comprising, in the case of a ski area, the portable apparatus has a display apparatus, a component for output of acoustic signals, and a GPS module and is designed such that the portable apparatus is fastenable to one of a skis or a snowboard of the person, and the display apparatus is visible to the person while skiing, and when information generated by the server is not displayed by the display apparatus, the display apparatus displays at least one of a speed, an elevation, and a traveled meters in height.

21. The method according to claim 17, further comprising transmitting the information generated by the server which can effect a redirection of the persons as push information, by the radio standard used to detect the persons or by a WLAN, or as text messages to the mobile electronic device of the person.

22. The method according to claim 17, further comprising transmitting the information generated by the server which information can effect a redirection of the persons by control of dynamic context-sensitive billboards depending on the current position of a specific person or groups of persons.

23. The method according to claim 17, further comprising transmitting the information generated by the server which information can effect a redirection of persons either optically or by a multicolor LED arrangement embedded in a ground within a specific area by light projectors, to guide individual persons or groups of persons, depending on a position of the individual persons or the groups of persons.

24. The method according to claim 17, further comprising transmitting the information generated by the server which information can effect a redirection of the persons by microdrones which are permanently or dynamically assigned to a selected person or the groups of persons, and the microdrones either accompany the person or groups of persons for an entire day or are dynamically assigned to the person and group of persons, as needed, by linking the ID and position of the person or group of persons to the position of a microdrone, and the microdrones are equipped with acoustic and/or visual signaling elements.

25. The method according to claim 17, further comprising, in a case of a ski area comprising chairlifts, providing a display device, for each seat of the chairlift, by which messages are displayed to a respective person during a ride on the chairlift which can help redirect that person.

26. The method according to claim 17, further comprising, in a case of a ski area comprising chairlifts, providing a fragrancing device for each seat of the chairlift by which fragrances are sprayable that can help redirect that person, and if a display apparatus is provided for each seat, and the fragrances correspond to the messages displayed via the display apparatus.

27. The method according to claim 17, further comprising, in a case of a ski area comprising lifts and an access control apparatus to the lifts, providing display apparatus, at each access control apparatus to the lifts of the ski area, by which messages are displayed to a person standing in front of the display apparatus that can effect the redirection of that person.

28. The method according to claim 17, further comprising wherein the content of the messages that can effect a redirection of persons depending on the current position of the persons, the current use of the facilities, and by an analysis of the current and if available of known older motion profiles of individual persons as well as a totality of persons is determined with reference to the people map, and a motion profile contains the average speed and direction of motion at places that require a decision regarding the direction of motion both with respect to individual person and group of persons, and a motion profile optionally contains durations of stays of individual persons or group of persons at specific points, optionally person-specific information is assigned to persons, which is obtained by assessing the images of 3D cameras or trinocular cameras, the motion profiles and, if available, the person-specific information about a person are assigned in the server to the unambiguous ID transmitted via the utilized radio standard, and thus to an unambiguous visitor profile.

29. The method according to claim 28, further comprising assessing the motion profiles and, if available, the person-specific information in order to predict the future motion profile of persons and group of persons for a specific predetermined time interval, and the motion profiles for the person and/or group of persons are obtained during the current visit of person or group of persons, and a quality of the data is improved over time during the visit day, and
taking action, depending on results of the assessment of the motion profiles, to avoid formation of queues, where the corresponding messages are transmitted to one or more of the individual persons.

30. The method according to claim 28, further comprising wherein if an already available user profile can be assigned to a detected person with reference to ID transmitted by the mobile electronic device of the person or with reference to the data of 3D cameras or trinocular cameras, testing in the server whether motion profiles exist for this visitor profile from a long-term past, and, if this is the case, testing whether a motion profile exists from a recent past, and, if a motion profile exists from a current day, testing whether an assessable context is available from the current visit, and, if this is not the case, combining motion profiles from the long-term past and recent past to make a prediction for a near future, that is, for a specific predetermined time interval in the future, and, if an assessable context does exist from the current visit, this is combined with the motion profiles from the recent past and long-term past to improve the prediction, if no motion profile exists from the recent past and an assessable context from the current visit exists, the motion profiles from the long-term past are combined with the assessable context to make the prediction, and, if no motion profile from the recent past and no assessable context from the current visit exist, the motion profiles from the long-term past are used to make the decision, and, if no motion profile from the long-term past exists, and a motion profile from the recent past and/or an assessable context exist, the available information is used for a prediction of the short-term future, and, if no motion profile exists from the long-term and recent past, the current position information is stored for updating the motion profile information, and, if no visitor profile can be assigned to a detected person, a new visitor profile is created and applied to this person, and this visitor profile is attached to the ID of the mobile electronic devices that the person carries with them and/or to the images of the 3D cameras or trinocular cameras.
